(19) United States
(12) Patent Application Publication
(10) Pub. No.: US 2017/0232360 A1
(43) Pub. Date: Aug. 17, 2017

(54) ALKALINE EARTH METAL SALTS

(71) Applicant: Imerys USA, Inc., Roswell, GA (US)

(72) Inventors: Miguel Angel VARGAS VELSCO, Cuautitlan Izacalli, C.P. (MX); Flor de Maria Montiel GONZALEZ, Coacalco de Berriozabal, C.P. (MX); Victor Leon RAMIREZ, Ecatepec, C.P. (MX); Dulce Jazmin OCAMPO SANTIAGO, Nezahualcoyotl, C.P. (MX); Gustavo Rodrigo MANCILLA SANCHEZ, San Antonio, C.P. (MX)

(21) Appl. No.: 15/518,630

(22) PCT Filed: Oct. 15, 2015
(86) PCT No.: PCT/US15/55759

§ 371 Date: Apr. 12, 2017

Related U.S. Application Data
(60) Provisional application No. 62/118,731, filed on Feb. 20, 2015.
(60) Provisional application No. 62/064,863, filed on Oct. 16, 2014.

(51) Int. Cl. B01D 1/18; C07C 51/41; A61K 9/20; A23L 33/16; A61K 8/365; A61Q 19/00; A23P 10/28; C07C 59/265; A61K 33/06

(52) U.S. Cl. CPC B01D 1/18; C07C 59/265; C07C 51/418; A61K 9/2095; A61K 33/06; A61K 8/365; A61Q 19/00; A23P 10/28; A23L 33/16; A61K 2800/28; A23V 2002/00

(57) ABSTRACT

A composition may include an alkaline earth metal salt. The alkaline earth metal salt may have an angle of repose less than or equal to about 34. The alkaline earth salt may be used to form a tablet. The tablet may have a friability less than or equal to about 1%. A method of forming an alkaline earth metal salt may include providing a slurry that includes an alkaline earth metal and an organic polyatomic anion, and spray drying the slurry to form a spray dried alkaline earth metal salt. The slurry temperature may be greater than or equal to about 50° C. A method of forming a tablet may include tableting a non-agglomerated alkaline earth metal salt.

### CLAIM FOR PRIORITY

This PCT International Application claims the benefit of priority of U.S. Provisional Patent Application Nos. 62/064,863, filed Oct. 16, 2014, and 62/118,731, filed Feb. 20, 2015, the subject matter of both of which is incorporated herein by reference in their entireties.

### FIELD OF DISCLOSURE

This disclosure relates to compositions of alkaline earth metal salts and methods of making an alkaline earth metal salts.

### BACKGROUND OF THE DISCLOSURE

Alkaline earth metals, such as calcium and magnesium, are important to proper nutrition. Calcium,for example, is important for bone and tooth formation and for proper muscular contraction, blood clotting, and nerve transmission functions. Magnesium, for example, is also important for maintaining normal nerve and muscle functions and for regulating blood glucose levels.

Calcium and/or magnesium deficiencies can lead to health problems. For example, low calcium can contribute to osteoporosis, which contributes to bone loss in men and women. Between ages 45 and 70, men and women may lose between 15 and 30% of their skeletal mass, with osteoporosis contributing to this loss The loss of skeletal mass and osteoporosis may contribute to fractures or replacement surgeries, such as hip replacement surgeries.

Calcium or magnesium supplements can be provided in the form of pills or tablets. However, these conventional formulations can be undesirable because the calcium- and magnesium-based powders are not easily compressible and require the addition of various additives, which reduces the amount of calcium or magnesium provided in the supplement. Processing methods may also require additional steps, such as a powder agglomeration step, to form tablets, which increases the cost and/or complexity of preparing the tablets. Furthermore, conventional calcium and magnesium supplements can have undesirable dissolution properties, resulting in a chalky or gritty texture due to poor dissolution of the tablet or pill. Tablet formulations can also be unduly soft, leading to fracture or breaking of the tablets.

Therefore, it may be desirable to provide an improved alkaline earth metal salt for use in tableting. It may also be desirable to provide tablets including an alkaline earth metal salt having high purity. It may also be desirable to provide a soluble alkaline earth metal salt in tablet or pill form.

### SUMMARY OF THE DISCLOSURE

In the following description, certain aspects and embodiments will become evident. It should be understood that the aspects and embodiments, in their broadest sense, could be practiced without having one or more features of these aspects or embodiments. It should be understood that these aspects and embodiments are merely exemplary.

According to an aspect of this disclosure, a composition may include an alkaline earth metal salt. According to another aspect, the alkaline earth metal salt may have an angle of repose less than or equal to about 34. According to another aspect, the alkaline earth metal salt may include a spray dried alkaline earth metal salt.

According to another aspect, a dispersible alkaline earth metal salt may include about a stoichiometric ratio of an alkaline earth metal and an organic polyatomic anion.

According to a further aspect, a tablet may include an alkaline earth metal salt. The alkaline earth metal salt may include an alkaline earth metal and an organic polyatomic anion. According to another aspect, the tablet may have a friability less than or equal to about 1%. According to another aspect, the alkaline earth metal salt may include a spray dried alkaline earth metal salt.

According to still another aspect, a method of forming an alkaline earth metal salt may include providing a slurry that includes an alkaline earth metal and an organic polyatomic anion, and spray drying the slurry to form a spray dried alkaline earth metal salt. According to yet another aspect, the slurry temperature may be greater than or equal to about 38° C.

According to another aspect, a method of forming a tablet may include providing a non-agglomerated alkaline earth metal salt and tableting the non-agglomerated alkaline earth metal salt.

According to still another aspect, a personal care composition may include an exfoliant that includes a spray dried alkaline earth metal salt.

### DESCRIPTION OF EXEMPLARY EMBODIMENTS

Reference will now be made in detail to exemplary embodiments.

Although certain embodiments may be discussed in terms of calcium citrate, it is understood that the discussion is used merely to facilitate understanding, and that other alkaline earth metal salts could be used in conjunction with the processes describe herein. Similarly, although certain embodiments may be discussed in terms of tablets or tableting processes, it is understood that the discussion is used merely to facilitate understanding, and that forming processes, such as, for example, pill forming processes, could be used in conjunction with the compositions and processes describe herein. As used in this disclosure, “alkaline earth metal” refers to group II metals (e.g., calcium and magnesium) and to transition metals having similar chemical properties, such as, for example, zinc, and combinations thereof.

According to some embodiments, a composition may include an alkaline earth metal salt. According to some embodiments, the alkaline earth metal salt may have an angle of repose less than or equal to about 34. According to some embodiments, the alkaline earth metal salt may include a spray dried alkaline earth metal salt.

According to some embodiments, a dispersible alkaline earth metal salt may include about a stoichiometric ratio of an alkaline earth metal and an organic polyatomic anion. According to some embodiments, the dispersible alkaline earth metal salt may include a crystalline alkaline earth metal salt. According to some embodiments, the dispersible alkaline earth metal salt may include a spray dried alkaline earth metal salt. According to some embodiments, the dispersible alkaline earth metal salt may include a soluble alkaline earth metal salt.

According to some embodiments, a tablet may include an alkaline earth metal salt. The alkaline earth metal salt may include an alkaline earth metal and an organic polyatomic anion. According to some embodiments, the tablet may have a friability less than or equal to about 1%. According to some embodiments, the alkaline earth metal salt may include a spray dried alkaline earth metal salt.

According to some embodiments, a method of forming an alkaline earth metal salt may include providing a slurry that includes an alkaline earth metal and an organic polyatomic anion, and spray drying the slurry to form a spray dried alkaline earth metal salt. According to some embodiments, the slurry temperature may be greater than or equal to about 38° C. According to some embodiments, the method may further include forming a tablet that includes the spray dried alkaline earth metal salt. According to some embodiments, forming the tablet may include a high-speed tableting process.

According to some embodiments, a method of forming a tablet may include providing a non-agglomerated alkaline earth metal salt and tableting the non-agglomerated alkaline earth metal salt. According to some embodiments, the tableting may include a high-speed tableting process.

According to some embodiments, a personal care composition may include an exfoliant that includes a spray dried alkaline earth metal salt.

According to some embodiments, the alkaline earth metal salt may include a powder or particulate alkaline earth metal salt.

According to some embodiments, an alkaline earth metal salt may include a spray dried alkaline earth metal salt. Spray drying is a method of producing a powder from a liquid or slurry by rapidly drying the liquid portion with a hot gas. Spray drying may include spraying small droplets of a slurry into a current of hot air in a large chamber to promote rapid evaporation of water from the droplets to produce a powder or particles. According to some embodiments, spray drying may produce a narrow or more consistent particle size distribution. In an exemplary spray drier, heated air acts as a drying medium and the slurry passes through an atomizer or spray nozzle(s) to disperse the liquid or slurry into a spray. The spray is then dried by the heated air, forming a powder or particles of the solids component of the slurry. Exemplary spray driers may include, but are not limited to, rotary disks, single-fluid high pressure swirl nozzles, and atomizer wheels. According to some embodiments, the spray drying may include a batch spray drying process. According to some embodiments, they spray drying may include a continuous spray drying process. According to some embodiments, the spray drier may be a vertical spray drier. According to some embodiments, the spray drier may be a horizontal spray drier.

According to some embodiments, the slurry or liquid for spray drying the alkaline earth metal salt may be prepared by creating a mixture that includes an alkaline earth metal component and an anionic component. The alkaline earth metal component may be formed from one or more alkaline earth metal compositions or precursors, such as, for example, an alkaline earth metal oxide, alkaline earth metal hydroxide, or alkaline earth metal carbonate. According to some embodiments, the alkaline earth metal composition may include a calcium-containing composition, a magnesium-containing composition, or a zinc-containing composition, such as, for example, calcium hydroxide, calcium oxide, calcium carbonate, magnesium hydroxide, magnesium oxide, or magnesium carbonate. The anionic component may be formed from an acid. The acid may include, according to some embodiments, an organic polyatomic acid, such as, for example, citric acid, malic acid, lactic acid, or tartaric acid. In solution, the anionic component of the acid may form an anion, such as, for example, a citrate, malate, lactate, or tartrate ion.

Without wishing to be bound to a particular theory, it is believed that the mixture of the alkaline earth metal composition and the acid may create a neutralization mixture, such that the alkaline earth metal and the anionic component of the acid interact to facilitate formation of the alkaline earth metal salt. According to some embodiments, the mixture may form a slurry of the alkaline earth metal salt or a slurry of the alkaline earth metal and the anionic component. For example, the slurry may contain a compound of the of the alkaline earth metal salt dispersed in the slurry, or may contain the alkaline earth metal and anionic component separately dispersed or dissolved within the slurry.

According to some embodiments, the slurry may include a solids content of the alkaline earth metal salt in a range from about 10% to about 40% by weight of the slurry, such as, for example, from about 20% to about 30% by weight of the slurry.

According to some embodiments, a binder may be added to the slurry. The binder may include a water soluble binder. Exemplary water soluble binders include, but are not limited to, starch, dextrose, polyvinyl alcohol, and vegetable gums. According to some embodiments, the binder may be added to the slurry in an amount in a range from about 0.01% to about 10% by weight relative to the alkaline earth metal salt. For example, the binder may be added in an amount in a range from about 0.01% to about 5%, from about 0.1% to about 10%, from about 0.01% to about 5%, from about 0.01% to about 3%, from about 0.01% to about 1%, from about 0.01% to about 0.1%, from about 1% to about 5%, from about 1% to about 3%, from about 0.01% to about 0.5%, from about 0.1% to about 5%, from about 0.1% to about 3%, from about 0.1% to about 1%, or from about 0.1% to about 0.5% by weight relative to the alkaline earth metal salt. According to some embodiments, the slurry may be mixed following the addition of the binder, which may facilitate dissolution of the binder.

According to some embodiments, the molar ratio of alkaline earth metal to anionic component in the slurry may be about a stoichiometric ratio. According to some embodiments, the molar ratio of alkaline earth metal to anionic component may vary from the stoichiometric ratio by ±5% of the alkaline earth metal, such as, for example, ±4% of the alkaline earth metal, ±3% of the alkaline earth metal, ±2% of the alkaline earth metal, or ±1% of the alkaline earth metal from the stoichiometric ratio. “Stoichiometric ratio,” as used in this disclosure, refers to the molar ratio of the constituents in a compound based on the charges of the constituents. For example, the stoichiometric ratio of calcium (Ca²⁺) and citrate ((C₆H₅O₇)³⁻) would be 3:2 (calcium:citrate).

The slurry may be spray dried to form a spray dried alkaline earth metal salt. The spray dried alkaline earth metal salt may include a powder or particulate alkaline earth metal salt.

According to some embodiments, the slurry temperature during the spray drying process may be greater than or equal to about 20° C., such as, for example, greater than or equal to about 30° C., greater than or equal to about 38° C., greater than or equal to about 40° C., greater than or equal to about 45° C., greater than or equal to about 50° C., greater than or equal to about 55° C., greater than or equal to about 60° C., greater than or equal to about 65° C., greater than or equal to about 70° C., greater than or equal to about 76° C., or greater than or equal to about 80° C.

According to some embodiments, the slurry temperature during the spray drying process may be in a range from about 20° C. to about 80° C., such as, for example, from about 38° C. to about 80° C., from about 40° C. to about 80° C., from about 40° C. to about 60° C., from about 50° C. to about 70° C., or from about 60° C. to about 80° C.

According to some embodiments, the temperature of the combustion chamber of the spray drier may be in a range from about 200° C. to about 400° C.

According to some embodiments, the alkaline earth metal salt, such as, for example, a spray dried alkaline earth metal salt, may include an alkaline earth metal and an polyatomic anion. According to some embodiments, an alkaline earth metal may include calcium, magnesium, barium, strontium, zinc, or a combination thereof. The polyatomic anion includes a polyatomic compound having an anionic (negative) charge. According to some embodiments, the polyatomic anion may be an organic polyatomic anion. Examples of organic polyatomic anions include, but are not limited to, citrates, malates, tartrates, lactates, and pharmaceutically or nutritionally acceptable salts or analogs thereof.

According to some embodiments, the alkaline earth metal salt may include calcium citrate, calcium malate, calcium tartrate, calcium lactate, magnesium citrate, magnesium malate, magnesium tartrate, magnesium lactate, zinc citrate, calcium citrate malate, calcium citrate lactate, calcium citrate tartrate, magnesium citrate malate, magnesium citrate lactate, magnesium citrate tartrate, pharmaceutically or nutritionally acceptable salts or analogs thereof, or combinations thereof.

As used herein, “pharmaceutically or nutritionally acceptable” refers to those compounds, materials, compositions, and/or dosage forms that are suitable for use in consumable products, medications, nutritional supplements, dietary supplements, and/or contact with the tissues of humans without excessive toxicity, irritation, allergic response, or other problem or complication commensurate with a reasonable benefit/risk ratio, and are effective for the intended use.

According to some embodiments, the alkaline earth metal salt may be a dispersible alkaline earth metal salt. “Dispersible,” as used in this disclosure, refers to the property of a substance, such as a salt, to disperse in an aqueous medium to form a suspension of the alkaline earth metal salt or to be soluble or dissolve in an aqueous medium, such as, for example, water. The dispersible substance may be broken down into one or more of its constituent ions, such as, for example, an cation and an anion or an alkaline earth metal and an anion, or may be dispersed as a compound having a cation chemically bound to an anion, such as, for example, by an ionic bond. According to some embodiments, the alkaline earth metal salt may be dispersible, such that greater than about 10 mg of calcium per fluid ounce of the alkaline earth metal salt are dispersed in an aqueous medium. According to some embodiments, the alkaline earth metal salt may be soluble in an aqueous medium. “Soluble,” as used in this disclosure, means that the alkaline earth metal salt is dispersed in water an amount to provide about 10 mg calcium per fluid ounce of the alkaline earth metal salt.

According to some embodiments, the alkaline earth metal salt may be an amorphous alkaline earth metal salt. According to some embodiments, the alkaline earth metal salt may be a dispersible, amorphous alkaline earth metal salt. According to some embodiments, the alkaline earth metal salt may be a soluble, amorphous alkaline earth metal salt. According to some embodiments, the alkaline earth metal salt may be an amorphous alkaline earth metal salt. According to some embodiments, the alkaline earth metal salt may be a semi-crystalline alkali earth metal salt.

As used herein, “amorphous” means non-crystalline (i.e., the lacks a distinct crystalline structure). Crystalline substances often contain relatively small amounts of non-crystalline matter and such structural defects do not detract from classification of these substances as crystalline since the physical properties of these substances are determined by their predominantly crystalline nature. Similarly, amorphous substances often contain small amounts of crystalline matter that do not significantly detract from their unique physical properties. “Semi-crystalline” refers to substances having both crystalline matter and amorphous matter in appreciable amounts such that both the crystalline and amorphous matter may affect the physical properties.

According to some embodiments, the molar ratio of alkaline earth metal to anionic component, such as, for example, organic polyatomic anion, may be about a stoichiometric ratio. According to some embodiments, the molar ratio of alkaline earth metal to anionic component may vary from a stoichiometric ratio by ±5% of the alkaline earth metal, such as, for example, ±4% of the alkaline earth metal, ±3% of the alkaline earth metal, ±2% of he alkaline earth metal, or ±1% of the alkaline earth metal from the stoichiometric ratio.

According to some embodiments, the alkaline earth metal salt may be characterized by an angle of repose. The angle of repose may describe the flowability of a material, such as, for example, a powder or particulate material. The angle of repose may be described by the acute angle formed between the side of a cone-shaped pile of a material and a horizontal surface upon which it rests. For example, the flatter the angle, the more flowable the material. Free flowing materials may generally have an angle of repose of less than 40 degrees, whereas materials which do not flow freely typically exhibit an angle of repose of 70 degrees or more.

The angle of repose may be measured by ASTM C-1444-00. This method includes placing a sample of material in a funnel with an opening large enough to let the largest particles of the sample through. The material under test is poured through the funnel onto a flat, solid surface and then, without shaking or vibrating the surface, measuring the angle the cone-like pile forms with the horizontal surface.

According to some embodiments, the alkaline earth metal salt may have an angle of repose less than or equal to about 40, such as, for example, less than or equal to about 36, less than or equal to about 34, less than or equal to about 32, less than or equal to about 30, less than or equal to about 28, less than or equal to about 26, less than or equal to about 24, or less than or equal to about 22. According to some embodiments, the alkaline earth metal salt may have an angle of repose in a range from about 20 to about 40, such as, for example, in a range from about 24 to about 34, from about 24 to about 30, or from about 26 to about 28.

According to some embodiments, the angle of repose may below enough that a desired bulk density of the alkaline earth metal salt is achieved, and may be sufficient to allow for the alkaline earth metal salt to flow through desired and/or necessary openings and channels during processing, such as, for example tableting or pill formation. According to some embodiments, the angle of repose may be sufficient to allow the alkaline earth metal salt to flow through processing equipment and to sufficiently fill molds or forms to form tablets or pills.

According to some embodiments, the alkaline earth metal salt may be characterized by the properties of particles or powder formed, such as, for example, during spray drying of the alkaline earth metal salt. According to some embodiments, the alkaline earth metal salt may be substantially free of agglomerated material.

According to some embodiments, the alkaline earth metal salt may be characterized by a median particle size. Median particle size (d₅₀) describes a property of the particles, where 50% of the particles have an equivalent spherical diameter less than or equal to the stated value. The median particle size may be determined by a Sedigraph Model 5120 III Particle Size Analyzer, as supplied by Micromeretics. The Sedigraph analysis determines particle size based on the distribution of particles using a laser light scattering and sedimentation technique.

According to some embodiments, the alkaline earth metal salt may have a median particle size less than or equal to about 100 microns. For example, the alkaline earth metal salt may have a median particle size less than or equal to about 80 microns, less than or equal to about 60 microns, less than or equal to about 50 microns, less than or equal to about 40 microns, less than or equal to about 30 microns, less than or equal to about 20 microns, or less than or equal to about 10 microns.

According to some embodiments, the alkaline earth metal salt may have a median particle size in a range from about 10 microns to about 100 microns. For example, the alkaline earth metal salt may have a median particle size in a range from about 20 microns to about 80 microns, from about 40 microns to about 80 microns, from about 50 microns to about 70 microns, from about 40 microns to about 60 microns, from about 20 microns to about 50 microns, from about 20 microns to about 40 microns, or from about 20 microns to about 30 microns.

According to some embodiments, the alkaline earth metal salt may be milled or ground after spray drying to reduce the size of the particles. For example, the alkaline earth metal salt may be milled to a median particle size less than or equal to about 30 microns, such as, for example, less than or equal to about 20 microns, less than or equal to about 10 microns, or less than or equal to about 8 microns.

According to some embodiments, the alkaline earth metal salt may be may be further subjected to an air sifter, hydrocyclone, or sieve to refine the distribution of particle sizes.

According to some embodiments, the particles or powder of the alkaline earth metal salt may be characterized by a sphericity of the particles. Sphericity may be observed by Scanning Electron Microscopy.

According to some embodiments, the alkaline earth metal salt may be formed into tablets or pills, such as, for example, dietary or nutritional tablets or pills, pharmaceutical tablets or pills, dietary or nutritional supplement tablets or pills. According to some embodiments, tablets or pills may be formed by providing the alkaline earth metal salt for filling a mold and then compressing the salt in the mold to form a pill or tablet. According to some embodiments, the tableting process may include a high-speed tableting process, such as, for example, a commercial scale tableting process. According to some embodiments, the tableting process may be carried out without first agglomerating the alkaline earth metal salt.

In tableting processes, to achieve acceptable tablets, powders have been agglomerated or granulated to facilitate formation of the tablets and to achieve desirable tablet properties. The alkaline earth metal salts, such as, for example, spray dried alkaline earth metal salts, described in this disclosure have been found to provide suitable tablet properties without first being agglomerated. The ability to form tablets and pills without an agglomeration or granulation step may reduce the cost of the tableting process. Forming tablets or pills without an agglomeration or granulation step may also simplify the tableting process, thereby increasing the efficiency with which the tablets can be produced.

According to some embodiments, tableting without an agglomeration or granulation step may allow for forming a higher-purity tablet, such as, for example, by reducing the amount of additives, such as additives to facilitate agglomeration or granulation, in the finished tablets or pills. Tableting using non-agglomerated particles may also facilitate dispersion or dissolution of the finished tablets. “Non-agglomerated” and similar phrases, as used in this disclosure, refer to a composition of alkaline earth metal salt particles or powder that is substantially free of granules in which the alkaline earth metal salt particles are collected into a larger particle with inter-particle bonds or binder. Exemplary agglomeration processes may include wet granulation or dry granulation processes.

According to some embodiments, a mold release agent may be used in the tableting process. The mold release agent may facilitate removal of the tablets from the tablet molds. Exemplary mold release agents include, but are not limited to, stearates, such as, for example, magnesium stearate.

According to some embodiments, the properties of tablets formed from an alkaline earth metal salt may be described in terms of the hardness or friability of the tablets. Friability may describe the property of a tablet to be reduced to smaller pieces and may provide an indication of the hardness of the tablet. Hardness or friability may be measured using friabilometer and hardnometer stokes.

According to some embodiments, tablets that include an alkaline earth metal salt may have a friability less than or equal to about 1%. For example, tablets that include an alkaline earth metal salt may have a friability less than or equal to about 0.8%, less than or equal to about 0.7%, less than or equal to about 0.5%, less than or equal to about 0.3%, less than or equal to about 0.2%, less than or equal to about 0.1%.

According to some embodiments, the tablets formed from an alkaline earth metal salt may be described in terms of the disintegration of the tablets in water. Disintegration of the tablets may be measured according to <701> disintegration of tablets, from the USP 37.

According to some embodiments, the tablet may have a dissolution time in water less than or equal to about 60 seconds, such as, for example, less than or equal to about 50 seconds, less than or equal to about 45 seconds, or less than or equal to about 40 seconds. According to some embodiments, the tablet may have a dissolution time in water in a range from about 30 seconds to about 60 seconds, such as, for example, from about 30 seconds to 50 seconds, or from about 40 seconds to about 50 seconds.

According to some embodiments, the tablet may include greater than or equal to about 80% by weight of alkaline earth metal salt relative to the weight of the tablet, such as, for example, greater than or equal to about 85% by weight, greater than or equal to about 90% by weight, greater than or equal to about 95% by weight, greater than or equal to about 97% by weight, greater than or equal to about 98% by weight, greater than or equal to about 99% by weight, greater than or equal to about 99.3% by weight, greater than or equal to about 99.5% by weight, greater than or equal to about 99.7% by weight, greater than or equal to about 99.8% by weight, greater than or equal to about 99.9% by weight, greater than or equal to about 99.95% by weight, greater than or equal to about 99.97% by weight, greater than or equal to about 99.98% by weight, or greater than or equal to about 99.99% by weight of alkaline earth metal salt relative to the weight of the tablet. According to some embodiments, the tablet may include about 100% by weight of alkaline earth metal salt relative to the weight of the tablet.

According to some embodiments, the tablet may include additives, such as, for example, agglomeration additives, dispersants, binders, lubricants, adsorbents, flavours, dyes, etc.

According to some embodiments, the tablet may include less than or equal to about 20% additives by weight of the tablet, such as, for example, less than or equal to about 15% additives by weight, less than or equal to about 10% additives by weight, less than or equal to about 5% additives by weight, less than or equal 3% additives by weight, less than or equal to about 2% additives by weight, less than or equal to about 1% additives by weight, less than or equal to about 0.8% additives by weight, less than or equal to about 0.5% additives by weight, less than or equal to about 0.3% additives by weight, less than or equal to about 0.2% additives by weight, less than or equal to about 0.1% additives by weight, less than or equal to about 0.05% additives by weight, less than or equal to about 0.03% additives by weight, less than or equal to about 0.02% additives by weight, or less than or equal to about 0.01% additives by weight of the tablet.

According to some embodiments, the tablet may contain substantially all alkaline earth metal salt. As used herein, “substantially all alkaline earth metal salt” is intended to mean only alkaline earth metal salt, taking into account normal or acceptable amounts of impurities from production and/or tableting processes.

According to some embodiments, an alkaline earth metal salt may be prepared by reacting an alkaline earth metal component with an acid in water. For example, calcium citrate may be prepared by reacting one or more of calcium hydroxide, calcium carbonate, or calcium oxide with citric acid in water. According to some embodiments, the ratio of the alkaline earth metal component to the acid may be provided in about a stoichiometric ratio of alkaline earth metal (e.g., alkaline earth metal cation) to the acidic anion of the acid. According to some embodiments, the ratio of the alkaline earth metal component to the acid may be provided in a range of about ±5% of the alkaline earth metal component, such as, for example, ±4% of the alkaline earth metal component, ±3% of the alkaline earth metal component, ±2% of the alkaline earth metal component, or ±1% of the alkaline earth metal component from the stoichiometric ratio of the alkaline earth metal to the acidic anion of the acid. Providing the alkaline earth metal component and the acid may facilitate a neutralization reaction in which the alkaline earth metal salt is formed. For example, one or more of calcium hydroxide, calcium carbonate, or calcium oxide may be provided with citric acid in water to form calcium citrate. Forming the alkaline earth metal salt in water may result in the formation of an alkaline earth metal salt slurry, for example, a slurry of calcium citrate.

According to some embodiments, a binder, such as, for example, starch, dextrose, polyvinyl alcohol, or vegetable gum, may be added to the alkaline earth metal salt slurry. According to some embodiments, the binder may include a water soluble binder. According to some embodiments, the binder may be added to the alkaline earth metal salt slurry in an amount in a range from about 0.01% by weight and 10% by weight based on alkaline earth metal salt. The binder-slurry composition may then be mixed to facilitate dissolution of the binder. According to some embodiments, the binder-slurry composition may be mixed for an amount of time sufficient to facilitate complete dissolution of the binder in the slurry, such as, for example, greater than or equal to about 30 minutes.

The alkaline earth metal salt slurry, either with or without the binder, may then be spray dried using any suitable form of spray drying equipment. The temperature and pressure of the spray drier may be selected as is appropriate for the application. According to some embodiments, the slurry temperature of the alkaline earth metal salt slurry at spray drying may be in a range from about 20° C. to about 80° C., such as, for example, in a range from about 40° C. to about 80° C. According to some embodiments, the pressure of the spray dryer may range from about 750 lbs/in² to about 1500 lbs/in². According to some embodiments, the temperature of the spray dryer may range from about 200° C. to about 400° C.

### EXAMPLE

A control calcium citrate was prepared by reacting calcium hydroxide and citric acid at a 3:2 stoichiometric (calcium:citrate) molar ratio in water to form a slurry of calcium citrate. The calcium citrate was isolated by passing the slurry over a drum filter. The calcium citrate was then flash dried and the temperature in the combustion chamber is of 400° C.

A sample spray dried calcium citrate was prepared by reacting calcium hydroxide and citric acid at a 3:2 stoichiometric (calcium:citrate) molar ratio in water to form a slurry of calcium citrate. The slurry was spray dried to form a spray dried calcium citrate powder.

Tablets were then formed from the control flash dried calcium citrate and the sample spray dried calcium citrate. The tablets were formed in compression molds at a Stokes B2 machine of sixteen stations that reaches a pressure of 18 to 20 kilograms per square centimeter.

The strength and friability of the tablets were measured using known equipment to measure friability and hardness tests, respectively. The flowability of each of the calcium citrate powders was visually evaluated for performance as either being suitable or not suitable.

The properties of the tablets were compared, and it was found that tablets made from the sample spray dried calcium citrate showed superior strength and lower friability when compared to tablets made from the control flash dried calcium citrate. The sample spray dried calcium citrate powder also had superior flowability compared to the control calcium citrate powder.

The improved flowability of the sample spray dried calcium citrate may be desirable in tableting processes, such as, for example, high speed tableting processes or commercial scale tableting processes. For example, improved flowability may result in better filling of the tablet molds during high speed or commercial scale tableting processes.

It was also determined that the sample spray dried calcium citrate was suitable for used in tableting to compress the powder and to form a high-purity tablet without the substantial addition of additives. The sample spray dried calcium citrate was also suitable for tableting without an agglomeration step.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

1. A composition comprising:
an alkaline earth metal salt having at least one of an angle of repose less than or equal to about 34.

2. The composition of claim 1, wherein the alkaline earth metal salt comprises a spray dried alkaline earth metal salt.

3. The composition of claim 1, wherein the alkaline earth metal salt comprises a dispersible, crystalline alkaline earth metal salt having about a stoichiometric ratio of a alkaline earth metal and an organic polyatomic anion.

4. The composition of claim 3, wherein the alkaline earth metal comprises calcium.

5. The composition of claim 3, wherein the organic polyatomic anion comprises citrate.

6. The composition of claim 1, wherein the alkaline earth metal salt has an angle of repose in a range from about 20 to about 34.

7. The composition of claim 1, wherein the alkaline earth metal salt has a median particle size less than or equal to about 100 microns.

8. (canceled)

9. The composition of claim 1, wherein the alkaline earth metal salt has a median particle size in a range from about 10 microns to about 100 microns.

10. The composition of claim 1, wherein a tablet comprising the alkaline earth metal salt has a friability less than or equal to about 1%.

11-24. (canceled)

25. A method of forming an alkaline earth metal salt, the method comprising: providing a slurry comprising an alkaline earth metal and an organic polyatomic anion; and spray drying the slurry to form a spray dried alkaline earth metal salt, wherein the slurry temperature is greater than or equal to about 38° C.

26. The method of claim 25, wherein the temperature of the slurry is greater than or equal to about 50° C.

27. The method of claim 25, wherein the slurry comprises about a stoichiometric ratio of the alkaline earth metal and the organic polyatomic anion.

28. The method of claim 25, wherein spray dried alkaline earth metal salt comprises a dispersible, crystalline alkaline earth metal salt.

29. The method of claim 25, wherein spray dried alkaline earth metal salt has a median particle size less than or equal to about 100 microns.

30. The method of claim 25, wherein spray dried alkaline earth metal salt has an angle of repose less than or equal to about 34.

31. The method of claim 25, further comprising:
forming a tablet comprising the spray dried alkaline earth metal salt.

32. The method of claim 31, wherein the spray dried alkaline earth metal salt used to form the tablet comprises a non-agglomerated spray dried alkaline earth metal salt.

33. The method of claim 31, wherein the tablet has a friability less than or equal to about 1%.

34. The method of claim 31, wherein the tablet comprises greater than or equal to about 95% by weight of the spray dried alkaline earth metal salt.

35. The method of claim 31, wherein the tablet has a dissolution time in water less than or equal to about 60 seconds.

36-45. (canceled)
